(19) United States
(12) Patent Application Publication
(10) Pub. No.: US 2013/0096417 A1
(43) Pub. Date: Apr. 18, 2013

(54) SETUP SHELL DEVICE, PATIENT TABLE COMPRISING A SETUP SHELL DEVICE, AND A MEDICAL IMAGING APPARATUS COMPRISING A PATIENT TABLE AND A SETUP SHELL DEVICE

(71) Applicant: DAGMAR HERING, Oberasbach (DE)

(72) Inventor: DAGMAR HERING, Oberasbach (DE)

(21) Appl. No.: 13/706,372

(22) Filed: Dec. 6, 2012

(30) Foreign Application Priority Data
Jun. 12, 2011 (DE) 102011087787.8

(51) Int. Cl. A61B 5/055

(52) U.S. Cl. CPC A61B 5/0555
USPC 600/415

(57) ABSTRACT

A setup shell device for setting up and/or positioning young children and/or babies for a medical imaging examination, including a body setup unit for accommodating and/or setting up the body of a young child and/or baby and a head setup unit for accommodating and/or setting up the head of a young child and/or baby. The head setup unit has a width that is less than the width of the body setup unit.

### CROSS REFERENCE TO RELATED APPLICATIONS

This application claims priority of German Patent Office application No. 102011087787.9 DE filed Dec. 6, 2011. All of the applications are incorporated by reference herein in their entirety.

### FIELD OF INVENTION

The disclosure relates to a setup shell device for setting up and/or positioning young children and/or babies for in particular a medical imaging examination, comprising a body setup unit for accommodating and/or setting up a young child's and/or baby's body and a head setup unit for accommodating and/or setting up a young child's and/or baby's head.

### BACKGROUND OF INVENTION

Medical imaging examinations, such as a magnetic resonance tomography examination (MRI scan), for example, provide a simple means of seeing inside the human body. An MRI examination takes on average between 20 and 50 minutes, a patient having to lie completely still during this time, apart from scanning pauses. The number of MRI examinations is increasing and a fast and optimum workflow is required to meet the demand.

In addition to the MRI scanning, a large proportion of a planned examination time is taken up by secure and comfortable positioning and/or setting up of the patient on a patient table of a medical imaging apparatus. In the case of young children and/or babies, the time required for this increases still further, as the positioning of young children and/or babies is very laborious. For example, the local coils and/or positioning aids required for an MRI examination are dimensioned and/or designed for adults and are suitable only to a limited extent or not at all for young children and/or babies.

In order to nevertheless enable young children and/or babies to be examined using a medical imaging machine, sandbags have been used, for example, to stably position young children and/or babies. However, this kind of positioning is very individual-specific and only poorly reproducible, if at all, for follow-up examinations.

### SUMMARY OF INVENTION

A device allowing rapid and in particular reproducible setting up and/or positioning of young children and/or babies for medical imaging examinations is provided by the features set forth in the independent claims. Advantageous embodiments are described in the dependent claims.

A setup shell device for setting up and/or positioning young children and/or babies for in particular a medical imaging examination is provided. The setup shell device comprising a body setup unit for accommodating and/or setting up a young child's and/or baby's body and a head setup unit for accommodating and/or setting up a young child's and/or baby's head.

In an embodiment, the head setup unit has a width that is less than the width of the body setup unit, thereby ensuring in particular secure and comfortable setup and/or positioning of young children and/or babies. In particular, the positioning of young children and/or babies may be made reproducible, particularly in respect of follow-up examinations. Thus, in the case of a magnetic resonance tomography examination, for example, a young child and/or baby may first be positioned in the setup shell device and then together with the setup shell device on a patient table of a magnetic resonance apparatus. Said setup shell device has a shape which essentially corresponds to the body shape of young children and/or babies, so that the latter may always assume an identical position inside the setup shell device. The term ‘width’ of the head setup unit and/or of the body setup unit should be understood here as meaning in particular a dimension along one direction, said direction preferably being perpendicular to a long side of the body shell unit and/or of the setup shell device.

In an embodiment, the head setup unit may have maximum width of 20 cm. However, in another embodiment the head setup unit has a maximum width of 18 cm. In addition, an embodiment, the body setup unit may have a maximum width of 35 cm. However, in another embodiment the body setup unit has a maximum width of 30 cm. This enables a particularly compact and space-saving setup shell device to be achieved. Moreover, because of the compact and particularly space-saving design of the setup shell device, a young child and/or a baby may be placed together with the setup shell device into a local coil, e.g. a head coil, for a magnetic resonance tomography examination, for example, thus allowing positioning reproducibility to be achieved particularly for follow-up examinations.

In an embodiment, the head setup unit may have a raised edge and the body setup unit has a raised edge, the raised edge of the head setup unit being made higher than the raised edge of the body setup unit, thereby enabling a young child's and/or a baby's head to be set up and/or positioned in a particularly stable manner for examination purposes such as a head examination and preventing undesirable tilting of the head during the examination. Moreover, head positioning with the aid of sandbags may be dispensed with. Said raised edge of the head setup unit may have a height of approximately 15 cm, wherein a height of approximately 15 cm is in particular to be understood as meaning that the height also has a tolerance range of ±4 cm. However the tolerance range may be ±2 cm. The maximum height of the raised edge of the body setup unit may be ⅔ of the height of the raised edge of the head setup unit so as to enable the young child and/or baby to be placed and positioned quickly and comfortably therein in spite of the raised edge. In addition, the young child and/or baby may be set up and/or transported inside the setup shell device, securely protected against falling out of the setup shell device.

In an embodiment, the body setup unit has a bottom end facing away from the head setup unit, the body setup unit being open at said bottom end. In this context, an open bottom end is to be understood as meaning in particular that the body setup unit has no raised edge in this region, so that said bottom end is open in the longitudinal direction. In this way young children and/or babies that are taller than a long side of the setup shell device may also be set up and/or positioned in the setup shell device for in particular a medical imaging examination.

In an embodiment, the body setup unit has a length of approximately 50 cm. However, in the body setup unit may have a length of approximately 45 cm. In another embodiment the body setup unit has a length of approximately 40 cm. In an embodiment, an approximation of the length is to be understood as meaning that the length of the body setup unit has a tolerance range of ±4 cm. In another embodiment the tolerance is ±2 cm. Thus a compact and space-saving setup shell device to be provided for setting up and/or positioning young children and/or babies is provided. In addition, the setup shell device may be made particularly user-friendly in this way.

In an embodiment, the body setup unit and the head setup unit are made of foamed plastic, thereby enabling the head setup unit and the body setup unit to be made from a soft and warm material of sufficiently high strength. In particular, the use of a foamed plastic for the body setup unit and the head setup unit enables the body heat of the young child and/or baby, particularly of a neonate, to be retained during the examination, such as an MRI scan, and helps to prevent undesirable cooling of the young child and/or baby, as could occur, for example, when using a smooth PVC shell as a setup shell device. In addition, it enables a particularly lightweight setup shell device to be provided, so that the setup shell device together with the young child and/or baby may be transported by a female medical technician. Alternatively, the body setup unit and/or the head setup unit may also be made from other materials deemed to be advisable by the average person skilled in the art, said materials having the property of softness for setting up and/or positioning the young child and/or baby while nevertheless providing a high degree of stability for transporting the young child and/or baby.

In an embodiment, the body setup unit may have a fastening unit for securing the young child and/or baby, thereby enabling particularly secure setting up and/or transporting of the young child and/or baby to be achieved using the setup shell device. The fastening unit may have two or more fastening straps which may be interconnected e.g. by means of Velcro strips, for safely setting up or rather securing the young child and/or baby. In addition, the fastening unit is of magnetic resonance compatible design, thereby also enabling the setup shell device to be used for magnetic resonance tomography examinations. Alternatively or additionally, the head setup unit may also have securing elements and/or restraining elements, such as fixatures, so that in particular the young child's and/or baby's head may be securely set up and restrained for a medical examination, which may be useful in the case of small infants and/or newly born babies, for example.

In another embodiment, the setup shell device has at least one fastening element for securing the setup shell device on a patient tabletop, thereby enabling the young child and/or baby to be placed in a reproducible position on the patient tabletop by means of the setup shell device particularly for medical imaging examinations. In addition, the fastening element may be implemented correspondingly to a fastening element encompassed by the patient tabletop, such as a fastening element implemented as a Velcro fastening, for example.

In addition, the setup shell device may also have a marking element whereby a location and/or a position of the setup shell device on a patient table of a medical imaging machine may be precisely recorded. Said marking element is preferably fixedly positioned on the setup shell device.

The setup shell device may be of fully magnetic resonance compatible design so that the setup shell device may be used for examinations using magnetic resonance equipment. For example, the setup shell device, in particular the head setup unit and the body setup unit, may be made of foamed plastic.

A patient table comprising a patient tabletop and a setup shell device for setting up and/or positioning young children and/or babies, particularly for a medical imaging examination is provided. The patient table allows the support shell unit to be positioned on the patient tabletop and the support shell unit provides a head setup unit for accommodating and/or setting up a young child's and/or baby's head and a body setup unit for accommodating and/or setting up a young child's and/or baby's body, and the head setup unit has a width that is less than the width of the body setup unit. The patient table provides a secure and a reproducible setting up and/or positioning of young children and/or babies. Thus, for magnetic resonance tomography examinations, for example, a young child and/or baby may first be positioned in the setup shell device and then positioned together with the setup shell device on a patient table of a magnetic resonance apparatus. Said setup shell device has a shape which essentially corresponds to the body shape of young children and/or babies, so that the latter may always assume an identical position within the setup shell device.

In an embodiment the patient table has a fastening unit for fastening the setup shell device to the patient tabletop, so that reproducible positioning of the young child and/or baby on the patient tabletop may be achieved. Both the setup shell device and the patient tabletop may each have a fastening element, wherein the two fastening elements may be implemented in a mutually corresponding manner, such as fastening elements comprising Velcro strips, for example, although an alternative design of the fastening unit is always possible. The fastening unit is preferably designed for detachable fastening of the setup shell device to the patient tabletop, so that the setup shell device may be removed from the patient tabletop at any time on completion of a scan.

Alternatively or in addition to a fastening unit, the patient table may also have a marking unit for positioning the setup shell device on the patient tabletop, by means of which a location and/or a position of the setup shell device on the patient tabletop of a medical imaging machine may be recorded. Said marking unit may comprise one or more marking elements which may be disposed on the setup shell device and/or on the patient tabletop.

A medical imaging apparatus having a patient table and a setup shell device is further provided.

In an embodiment, the setup shell device may be positioned for setting up a young child and/or baby on the patient table for a medical imaging examination of the young child and/or baby. The use of the setup shell device provides access to the young child and/or baby e.g. for observation during a medical imaging examination. In addition, the workflow for medical imaging examinations may be improved by preparing the young children and/or babies for the medical imaging examination in an adjacent room, for example. This may include in particular setting up and/or positioning on the setup shell device and/or attaching respirators and/or monitoring devices to the young child and/or baby. In this way the young children and/or babies may be being prepared even while a preceding medical imaging examination is in progress, thus enabling significant time savings to be achieved for medical imaging examinations.

In an embodiment the setup shell device may be positioned together with a local coil unit on the patient table. The local coil unit, e.g. a head coil designed for adult patient use, may also be used particularly simply for examinations of young children and/or babies, and reproducible positioning of the young child and/or baby with respect to the local coil may be achieved by, for example, inserting the setup shell device together with the young child and/or baby into the local coil. In addition, the use of sandbags to position the young child and/or baby inside the local coil may be dispensed with.

### BRIEF DESCRIPTION OF THE DRAWINGS

Features and details will emerge from the embodiment described below and with reference to the accompanying drawings in which:

FIG. 1 schematically illustrates a setup shell device,

FIG. 2 shows a side view of the setup shell device from FIG. 1,

FIG. 3 shows a plan view of the setup shell device from FIG. 1 and

FIG. 4 schematically illustrates an inventive magnetic resonance apparatus incorporating the setup shell device.

### DETAILED DESCRIPTION OF INVENTION

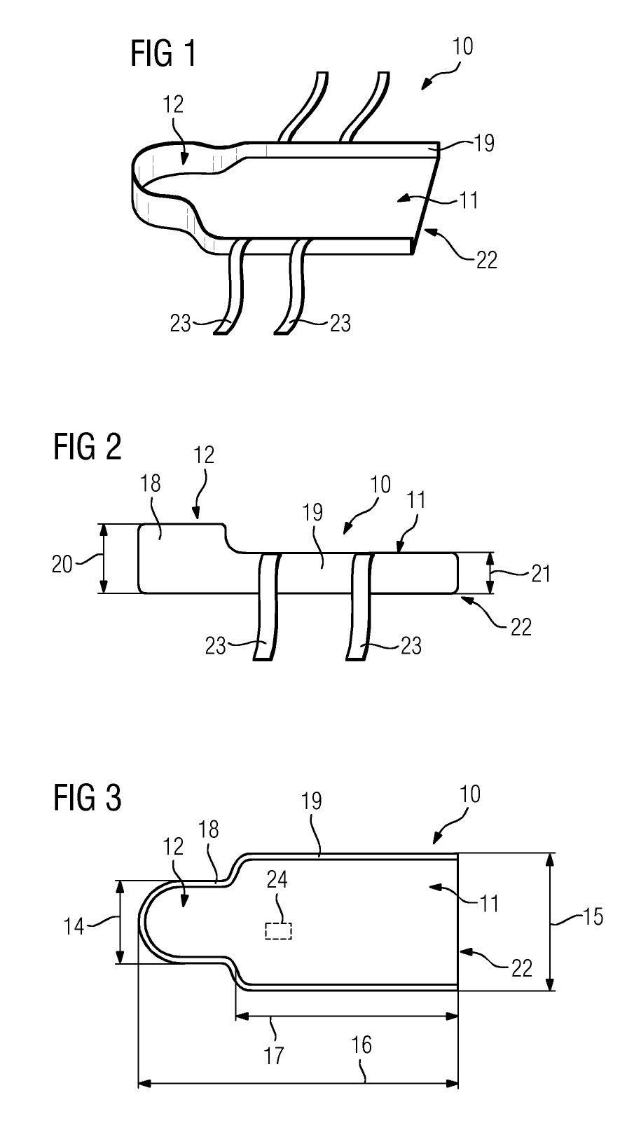

FIGS. 1 to 3 schematically illustrate a setup shell device 10 for setting up and/or positioning young children and/or babies 13. The setup shell device 10 has a body setup unit 11 for accommodating and/or setting up the body of a young child and/or baby 13 and a head setup unit 12 for accommodating and/or setting up the head of a young child and/or baby 13.

The shape of said setup shell device 10 is essentially modeled on the body shape of young children and/or babies 13. The head setup unit 12 may have a width 14 of max. 20 cm so that the head of a young child and/or baby 13 may be comfortably and in particular securely supported to prevent it from turning and/or tilting. However, the head setup unit 12 may have the width 14 of max. 18 cm. The width 14 of said head setup unit 12 is less then the width 15 of the body setup unit 11. For example, the body setup unit 11 may have a width 15 of max. 35 cm or max. 30 cm. The width 14 of the head setup unit 12 and the width 15 of the body setup unit 11 are in a direction perpendicular to the long side 16 of the setup shell device 10 (FIG. 3). The body shell unit 11 has a length of approximately 50 cm, wherein the length is in the direction of the long side 16 of the setup shell device 10.

In addition, the head setup unit 12 has a raised edge 18 and the body setup unit 11 a raised edge 19, wherein the raised edge 18 of the head setup unit 12 is made higher than the raised edge 19 of the body setup unit 11 (FIGS. 1 and 2). The raised edge 18 of the head setup unit 12 has a height 20 of approximately 15 cm and the raised edge 19 of the body setup unit 11 has a height 21 of max. 10 cm, so that the young child and/or baby 13 may be securely supported in the setup shell device 10 to prevent said young child and/or baby from falling out, for example, when the young child and/or baby 13 is being transported using the setup shell device 10.

The raised edge 19 of the body shell unit 11 is interrupted at a bottom end 22 of the body setup unit 11 facing away from the head setup unit 12 so that the body shell unit 11 is open at this end. This makes it possible for young children and/or babies 13 exceeding the length of the setup shell device 10 to be set up in the setup shell device 10. Alternatively, the body setup unit 11 may also be provided with a raised edge at the bottom end 22.

In addition, to secure and/or fasten the young child and/or baby 13, the body setup unit 11 has a fastening unit 23 which in this exemplary embodiment has four fastening straps. The fastening straps are fixedly disposed on the body shell unit 11. To fasten the young child and/or baby 13, the fastening straps have Velcro strips. In addition, alternative designs of the fastening unit 23 are always possible. Moreover, the head setup unit 12 may also have securing elements and/or fixing elements not shown in greater detail, e.g. fixatures, which are designed in particular for securely fixing and/or setting up the head of the young child and/or baby 13. By means of such securing elements and/or fixing elements, the head of a young baby 13 and/or newly born baby may be securely and in particular reproducibly positioned inside the setup shell device 10 for an examination, in particular a medical imaging examination.

The setup shell device 10 is of completely magnetic resonance compatible design for use in conjunction with a medical imaging apparatus constituted by a magnetic resonance apparatus 100. For this purpose the body setup unit 11 and the head setup unit 12 are made of foamed plastic. In addition, the fastening unit 23 is also made from a magnetic resonance compatible material.

If the setup shell device 10 is not intended to be used in conjunction with a magnetic resonance apparatus 100, the setup shell device 10 may also be made from materials that are not magnetic resonance compatible.

For positioning the setup shell device 10 on a patient table 101, in particular a tabletop 102 of the patient table 101, the setup shell device 10 has a fastening element 24 consisting, for example, of Velcro strips. This fastening element 24 is disposed on the underside, facing away from the accommodation area for accommodating the young child and/or baby 13, of the body setup unit 11 (indicated by dashed lines in FIG. 3). Other types of fastening element 24 that appear practical to the average person skilled in the art are always possible.

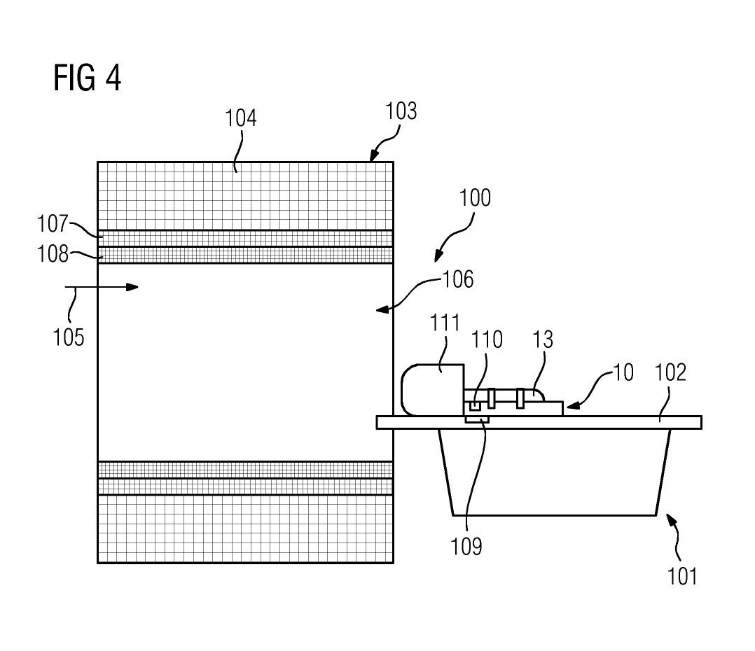

FIG. 4 schematically illustrates the medical imaging apparatus which is constituted, by way of example, by a magnetic resonance apparatus 100. The magnetic resonance apparatus 100 comprises a magnet unit 103 having a main magnet 104 for producing a powerful and in particular constant main magnetic field 105. The magnetic resonance apparatus 100 additionally has a cylindrical accommodation area 106 for accommodating a patient. The patient may be slid into the accommodation area 106 by means of the patient table 101.

The magnet unit 103 additionally has a gradient coil 107 for producing magnetic field gradients which is used for local coding during imaging. In addition, the magnet unit 103 has an RF antenna 108. The RF antenna 108 beams RF magnetic resonance frequencies into an examination space which is essentially constituted by the accommodation area 106.

The patient table 101 of the magnetic resonance apparatus 100 comprises the patient tabletop 102 and the setup shell device 10. For a magnetic resonance tomography examination of the young child and/or baby 13, the setup shell device 10 is positioned on the patient tabletop 102, as shown in greater detail in FIG. 4. For positioning the setup shell device, the patient table 101 has a fastening unit comprising a fastening element 109 which is made to correspond to the fastening element 24 of the setup shell device 10 and is encompassed by the patient tabletop 102. Alternatively, only the setup shell device 10 or the patient table 101 may have a fastening element 24, 109. In addition, an alternative design of the fastening unit is also possible.

In addition, the patient table 101 also has a marking unit by means of which the positioning of the setup shell device 10 on the patient tabletop 102 may be precisely recorded. A marking element 110 of the marking unit is disposed on the setup shell device, said marking element 110 being disposed on the raised edge 19 of the body shell unit 11. The marking element 110 may be used to record precisely the position of the setup shell device 10 on the patient tabletop 102. Moreover, it is also conceivable for the patient tabletop 102 to have a marking element for positioning the setup shell device 10.

For a magnetic resonance tomography examination, the young child and/or baby 13 is first positioned and/or set up inside the setup shell device 10. Additional units for monitoring the young child and/or baby 13 such as respiration accessories and/or monitoring accessories, etc. are also often attached to the young child and/or baby 13. The setup shell device 10 together with the therein positioned young child and/or baby 13 is then positioned on the patient tabletop 102 of the patient table 101 in an examination position. If local coils 111 are additionally required for the magnetic resonance tomography examination in question, such as a head coil for a head examination, for example, this is attached to the young child and/or baby 13, said young child and/or baby 13 lying on the setup shell device 10 being placed e.g. in a head coil together with the head support area 12 of the setup shell device 10. For additional coils, it may also be provided that the setup shell device 10 with the young child and/or baby 13 retains its position on the patient tabletop 102 after positioning and the local coil is positioned around the young child and/or baby 13 and the setup shell device 10. This always ensures optimum positioning of the young child and/or baby 13 with respect to the local coil 111.

While specific embodiments have been described in detail, those with ordinary skill in the art will appreciate that various modifications and alternative to those details could be developed in light of the overall teachings of the disclosure. For example, elements described in association with different embodiments may be combined. Accordingly, the particular arrangements disclosed are meant to be illustrative only and should not be construed as limiting the scope of the claims or disclosure, which are to be given the full breadth of the appended claims, and any and all equivalents thereof. It should be noted that the term “comprising” does not exclude other elements or steps and the use of articles “a” or “an” does not exclude a plurality.

1. A setup shell device for setting up and/or positioning young children and/or babies in a medical imaging examination, comprising:
a body setup unit for accommodating and/or setting up the body of a young child and/or baby; and
a head setup unit for accommodating and/or setting up the head of a young child and/or baby,
wherein the head setup unit has a width that is less than the width of the body setup unit.

2. The setup shell device as claimed in claim 1,
wherein the head setup unit has a maximum width of 20 cm.

3. The setup shell device as claimed in claim 1,
wherein the body setup unit has a maximum width of 35 cm.

4. The setup shell device as claimed in claim 1,
wherein the head setup unit includes a raised edge,
wherein the body setup unit includes a raised edge, and
wherein the raised edge of the head setup unit is higher than the raised edge of the body setup unit.

5. The setup shell device as claimed in claim 4,
wherein the raised edge of the head setup has a height of approximately 15 cm.

6. The setup shell device as claimed in claim 1,
wherein the body setup unit includes a bottom end facing away from the head setup unit, and
wherein the body setup unit is open at the bottom end.

7. The setup shell device as claimed in claim 1,
wherein the body setup unit has a length of approximately 50 cm.

8. The setup shell device as claimed in claim 1,
wherein the body setup unit and the head setup unit are made of foamed plastic.

9. The setup shell device as claimed in claim 1,
wherein the body setup unit includes a fastening unit for securing the young child and/or baby.

10. The setup shell device as claimed in claim 1, comprising:
a fastening element for securing on a patient tabletop.

11. The setup shell device as claimed in claim 1, comprising:
a marking element for recording a position during the medical imaging examination.

12. The setup shell device as claimed in claim 1,
wherein the setup shell device has a magnetic resonance compatible design.

13. A patient table, comprising:
an patient tabletop; and
a setup shell device as claimed in claim 1 which is positioned on the patient tabletop.

14. The patient table as claimed in claim 13, comprising:
a fastening unit for fastening the setup shell device to the patient tabletop.

15. The patient table as claimed in claim 13,
a marking unit for positioning the setup shell device on the patient tabletop.

16. The patient table as claimed in claim 14,
a marking unit for positioning the setup shell device on the patient tabletop.

17. A medical imaging apparatus, comprising:
a patient table as claimed in claim 13.

18. The medical imaging apparatus as claimed in claim 17,
wherein the setup shell device is positioned together with a local coil unit on the patient table.
